(19) United States
(12) Patent Application Publication
(10) Pub. No.: US 2001/0018424 A1
(43) Pub. Date: Aug. 30, 2001

(54) Novel family of compositions based on alkylpolyglycosides and dimerdiol, which are especially useful for the preparation of vaporisable oil-in-water emulsions

(76) Inventors: Alain Milius, Nice (FR); Jean-Pierre Boiteux, Saix (FR); Herve Rolland, Castres (FR); Guy Tabacchi, Castres (FR); Chantal Amalric, Blan (FR)

Correspondence Address: Timothy J. Keefer, Wildman, Harrold, Allen & Dixon, 225 West Wacker Drive, Chicago, IL, 60606, US

(21) Appl. No.: 09/782,775

(22) Filed: Feb. 13, 2001

(30) Foreign Application Priority Data
Feb. 14, 2000 (FR) 0001783

(51) Int. Cl. A61K 31/70; A61K 7/00; A61K 31/045; A61K 31/20; A61K 31/715

(52) U.S. Cl. 514/25; 514/53; 514/560; 514/738; 424/47; 424/401; 424/414

(57) ABSTRACT

The present invention relates to a novel family of compositions based on alkylpolyglycosides and dimerdiol, which are especially useful for the preparation of vaporisable oil-in-water emulsions.

According to the invention, these compositions comprise: 5 to 95 parts by weight of a mixture of alkylpolyglycosides which is constituted of the products of a reaction of a saccharide and a dimerdiol having 36 carbon atoms; 95 to 5 parts by weight of a dimerdiol having 36 carbon atoms.

The present invention relates to a novel family of compositions based on alkylpolyglycosides and dimerdiol, which are especially useful for the preparation of vaporisable oil-in-water emulsions.

The invention finds application mainly in the field of cosmetics, but its use can also be envisaged in the fields of pharmaceuticals (nasal spray, vaccine), plant treatment, paper treatment, textile treatment, as well as for household applications or industrial applications (lubricants, for example).

Within the context of the present description, the expression <<vaporisable emulsion>> refers to an emulsion which is stable in a very low viscosity zone, less than 3,000 cps, preferably between 50 and 3,000 cps, and more preferably between 100 and 1,500 cps. These emulsions are further referred to by the expression <<sprayable emulsions>>.

Alkylglycosides or alkylpolyglycosides (APGs) are well-known non-ionic surfactant compounds which can be used alone, or in combination with other surfactants, in a wide range of industrial applications and especially in the field of cosmetics.

Alkylpolyglycosides have first of all been used as foaming agents and in this application, those the alkyl chain of which comprises from 8 to 14 carbon atoms have proved to be particularly interesting.

More recently, alkylpolyglycosides have been used as emulsifiers, and in this application, those the alkyl chain of which comprises from 16 to 18 carbon atoms have proved to be particularly interesting.

The patent application WO 92/06778, in the name of the Applicant, describes, for the first time, the use of mixtures of alkylpolyglycosides and fatty alcohols as self-emulsifying agents.

The term <<self-emulsifying>> designates any agent or composition which is capable of forming a stable emulsion with an aqueous phase, practically without the provision of energy, for example by dispersion in the aqueous phase by slow mechanical agitation.

More specifically, the mixtures described in this prior art document comprise:

from 60 to 90% by weight of at least one fatty alcohol having from 12 to 22 carbon atoms, and preferably from 16 to 18 carbon atoms; and

from 10 to 40% by weight of an alkylpolyglycoside, the alkyl part of which is preferably identical to that of the fatty alcohol.

The self-emulsifiable compositions described in the above-mentioned application are marketed under the designation Montanov® 68 and comprise a mixture of alkylpolyglycosides the fatty chains of which comprise 16 and 18 carbon atoms, as well as a mixture of fatty alcohols of the same fatty chain length.

If such compositions are perfectly satisfactory, especially as regards the stability of the emulsions that they enable to obtain, they are solids and do not however enable fluid emulsions to be prepared easily.

Furthermore, the patent application WO 95/13863, in the name of the Applicant, describes compositions which are also based on alkylpolyglycosides and fatty alcohols, and which are presented in the form of concentrates which are especially useful for the preparation of fluid emulsions.

These compositions are essentially characterised by the fact that they comprise a mixture of at least two alkylpolyglycosides which differ by the nature of their alkyl part.

It is specified that at least one of these alkylpolyglycosides comprises an alkyl chain having from 16 to 22 carbon atoms, and preferably from 16 to 18 carbon atoms.

It is specified further that the alkylpolyglycosides comprising an alkyl part having from 16 to 22 carbon atoms must represent at least 25% of the mixture of alkylpolyglycosides, and in all the Examples, these alkylpolyglycosides comprise 16 or 18 carbon atoms and represent at least 50% by weight of the mixture of alkylpolyglycosides.

The compositions described in this document enable fluid emulsions of the oil-in-water type to be prepared, but they are in a solid form and, consequently, need to be heated in order to be able to be made use of.

In the FR 98 13255 document, a novel family of compositions based on alkylpolyglycosides and fatty alcohols is proposed which enable, at low doses, stable fluid emulsions to be prepared, without use of a co-surfactant or a stabiliser.

These compositions are notably characterised by the fact that they comprise a mixture, in well-defined proportions, of four types of alkylpolyglycosides which comprise an alkyl chain having 12, 14, 16 or 18, 20 or 22 carbon atoms, respectively.

A composition such as described in this prior art document is marketed under the designation Montanov® L and corresponds to the composition described in Example 1 of this document.

The compositions described in this document enable vaporisable emulsions of the oil-in-water type to be prepared, but they are in a solid form and, consequently, need to be heated in order to be able to be made use of.

In the document FR 99 03429 the use is proposed as emulsifier of oleylglycoside and isostearylglycoside in a mixture with oleic alcohol or isostearylic alcohol for the preparation of stable water-in-oil emulsions.

The compositions described in the document mentioned above are in a liquid form and due to this, they are particularly easy to make use of.

However, the compositions do not enable emulsions of the oil-in-water type to be obtained.

Under these circumstances, the aim of the present invention is to solve the technical problem consisting of providing novel liquid compositions based on alkylpolyglycosides and fatty alcohols, which allow the preparation of vaporisable oil-in-water emulsions.

The solution in accordance with the present invention for solving this technical problem consists of novel compositions based on alkylpolyglycosides and fatty diols, characterised in that they comprise:

5 to 95 parts by weight of a mixture of alkylpolyglycosides which is constituted of the products of a reaction of a saccharide and a dimerdiol having 36 carbon atoms;

95 to 5 parts by weight of a dimerdiol having 36 carbon atoms.

It has been discovered, and this constitutes the basis of the invention, that such compositions are in liquid form and enable oil-in-water emulsions to be obtained which have properties of stabilities in a very low viscosity zone, and which are particularly interesting, for their uses by vaporisation, especially in the field of cosmetics.

For example, it is possible for the compositions in accordance with the present invention to be used for the impregnation of wipes, as well as for the preparation of self-tanning lotions, solar products or after-sun products and generally for any application which necessitates a homogeneous spreading and/or a rapid penetration of an active substance.

The compositions which are preferred within the context of the present invention are the compositions which comprise:

5 to 60 parts by weight of the above-mentioned mixture of alkylpolyglycosides; and

95 to 40 parts by weight of dimerdiol having 36 carbon atoms.

The mixture of alkylpolyglycosides which is constituted of the products of a reaction of a saccharide and a dimerdiol having 36 carbon atoms is in fact constituted of a mixture of any proportion of hydroxyalkylpolyglycosides (products resulting from the acetalisation of one of the two hydroxyl groups of dimerdiol) and of polyglycosylalkylpolyglycosides (products resulting from the acetalisation of the two hydroxyl groups of dimerdiol).

These alkylpolyglycosides which can be represented, respectively, by the following formulae I and II:

HO—R—O(G)ₙ (I)

(G)ₘ—OR—O—(G)ₚ (II)

in which:

G represents a saccharide residue;

R represents a disubstituted group derived from the dimer alcohol originating from the hydrogenation from the dimer acid;

n, m and p represent the average degree of polymerisation of each saccharide residue.

The product known under the designation <<dimer acid>> is a dibasic acid having 36 carbon atoms the major compound of which can be represented by the formula:

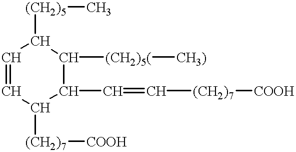

The alkylpolyglycosides mentioned above can contain, a glucose or dextrose, fructose, galactose, mannose, ribose, xylose residue, preferably a glucose residue, as saccharide residue.

It is to be further noted that each unit of the polyoside part of the alkylpolyglycosides mentioned above can be in α or β anomeric form, and the saccharide residue can be of furanoside or pyranoside type.

The average degree of polymerisation of each saccharide residue is generally between 1.05 and 2.5, more preferably between 1.1 and 2.

The expression <<alkylpolyglycoside>> used within the context of the present application therefore designates equally an alkylmonooside (degree of polymerisation equal to 1) or an alkylpolyglycoside (degree of polymerisation greater than 1).

The dimerdiol used for the preparation of the compositions in accordance with the present invention is a diol originating from the hydrogenation of the dimer acid.

It is notably marketed by the companies HENKEL or SIDOBRE-SINNOVA under the designation SPEZIOL® C36/2.

This compound, due to its origin, can contain minor proportions of impurities. Such impurities can be present in amounts ranging up to 30% by weight of the total weight of diol.

Consequently, the compositions in accordance with the present invention can comprise, in corresponding minor proportions, such impurities or the products of reaction of these impurities with a saccharide.

The compositions based on alkylpolyglycosides and dimerdiol in accordance with the present invention can be prepared by simple mixing of their constituents in pre-determined proportions desired.

On an industrial scale, they will preferably be prepared according to one of the two routes classically used for the synthesis of alkylpolyglycosides, and for example, by reaction in acid medium between the dimerdiol and a saccharide having an anomeric OH, such as glucose or dextrose.

Such synthetic routes are well known and have been described in numerous documents, and in particular in the documents of the Applicant mentioned above.

If need be, it will be possible for this synthesis to be completed by operations of neutralisation, filtration, distillation or partial extraction of excess fatty diol, or a discoloration operation.

The compositions based on alkylpolyglycosides and dimerdiol in accordance with the present invention can notably be used for the preparation of cosmetic, pharmaceutical, hygiene or detergent oil-in-water vaporisable emulsions.

Solar emulsions, perfume sprays, emulsions for impregnating wipes, emulsions for the preparation of moisturising handkerchiefs, and de-make-up products can be cited in particular amongst the vaporisable emulsions which can be prepared within the context of the present invention.

In general, such an emulsion will comprise from 1 to 25% by weight, preferably from 1 to 10%, and more preferably from 1 to 5% by weight of the composition based on alkylpolyglycosides and dimerdiol in accordance with the present invention.

Furthermore, it will be possible for these emulsions to comprise an oily phase.

The constituent oily phase of the emulsion can be constituted by free dimerdiol, constituent of the emulsifying composition of the invention, without it being necessary to make use of another oil. However, more generally, an oil will be used which is selected from the following oils:

oils of plant origin, such as sweet almond oil, coconut oil, castor oil, jojoba oil, olive oil, rapeseed oil, groundnut oil, sunflower oil, wheat germ oil, maize germ oil, soya oil, cotton oil, lucerne oil, poppy oil, marrow oil, evening primrose oil, millet oil, barley oil, rye oil, safflower oil, canelle nut tree oil, passionflower oil, hazelnut oil, palm oil, shea butter, apricot stone oil, Alexandria laurel tree oil, sysymbrium oil, avocado oil, calendula oil;

modified plant oils such as the products known under INCI designations Apricot Kernel Oil PEG-6 esters and Olive Oil PEG-6 esters;

oils of natural origin, such as perhydrosqualene, squalene;

mineral oils, such as liquid paraffin, and mineral oils, notably originating from petroleum fractions, such as isoparaffins, having a boiling point between 300 and 400° C.;

synthetic oils, notably fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, ester derivatives of lanolic acid, such as isopropyl lanolate, isocetyl lanolate, triglycerides such as glycerol triheptanoate, alkylbenzoates, isoparaffins, polyalphaolefins, polyolefins, synthetic isoalkanes such as isohexadecane, isododecane, and silicone oils. Amongst the latter oils, dimethyl polysiloxanes, methylphenylpolysiloxanes, amine-modified silicones, fatty acid-modified silicones, alcohol-modified silicones, alcohol- and fatty acid-modified silicones, polyether group-modified silicones, epoxy-modified silicones, fluoro group-modified silicones, cyclic silicones, and alkyl group-modified silicones, may be more particularly cited.

Generally, the emulsions in accordance with the present invention will comprise up to 50%, and preferably between 2 and 30% by weight of oily phase as defined above.

These emulsions can be prepared by simple dispersion of a fatty phase constituted of the composition according to the invention and optionally of one or more oils such as the oils described above, in a hydrophilic phase, generally water or a hydrophilic solvent.

The dispersion will preferably be done in the cold, all the constituents must be liquid at the time of mixing.

These emulsions can, further, comprise an additional emulsifying agent in an amount such that the total amount of emulsifying agents within the emulsion be less than or equal to 25% by weight.

Thus, according to a second aspect, the present application aims to cover vaporisable liquid emulsions of the oil-in-water type which comprise at least one aqueous phase and an oily phase and, as emulsifier, a composition based on alkylpolyglycosides and dimerdiol as defined above.

The emulsions which are particularly preferred within the context of the present invention will generally comprise:

from 1 to 10% by weight of a composition based on alkylpolyglycosides and dimerdiol as defined above;

from 0 to 50% by weight of oil; and

an aqueous phase.

The invention will be illustrated in detail by the following Examples, which are given solely as an illustration.

### EXAMPLE 1

### Method of Preparing a Composition in Accordance with the Invention by Direct Acetalisation

450 g of C₃₆ dimer alcohol (marketed by the company Sidobre-Sinnova under the designation SPEZIOL® C36/2) are introduced into a two-litre glass reactor equipped with an efficient mechanical stirrer, a system of heating by double envelope, a condenser and a temperature probe.

The dimer alcohol, which is in the form of a viscous liquid, is heated to 80° C. and 75 g of anhydrous dextrose are dispersed in the reaction medium and then homogenised at 80° C. for 15 minutes.

The temperature is then fixed at 105° C. and 2 g of 96% sulphuric acid are then added.

The reaction mixture is then maintained for 8 hours at 105° C. under partial vacuum and with nitrogen bubbling.

After cooling to 90° C., the reaction mixture is neutralised so as to obtain a pH value of a 5% solution of the reaction medium of around 6.2 by the addition of a solution of sodium hydroxide.

The resulting product is in the form of a viscous liquid having a free dimer alcohol content of 70% by weight.

### EXAMPLE 2

### Method of Preparing a Composition in Accordance with the Invention by Transetherification

2a) Preparation of Butylglucoside

900 g of butyl alcohol and 50 g of heptane are introduced into a two-litre glass reactor, equipped with a double envelope, an efficient mechanical stirrer, a thermometric probe and a Dean Stark type distillation system.

The medium is brought to 85° C., a temperature at which 360 g of anhydrous dextrose and 1.8 g of 96% sulphuric acid are added.

The mixture is then heated at 105- 110° C. for 4 hours with continuous recirculation of the heptane/butanol mixture which distils.

2b) Transetherification

625 g of the preceding reaction medium, containing 65 to 60% of residual butanol are heated to 85° C. and 1,140 g of C36 dimer alcohol (SPEZIOL® C36/2) are added in dispersion.

The mixture is then heated at 95° C. under partial vacuum for 5 hours with continuous distillation of the butyl alcohol.

The reaction medium is then cooled to 85° C., neutralised so as to obtain a pH value of a 5% solution of the reaction medium of between 5.5 and 7.5 with a sodium hydroxide solution.

The product obtained, which is liquid at ambient temperature, has a free dimer alcohol content of 50% by weight.

### EXAMPLE 3

### Method of Preparing a Composition in Accordance with the Invention by Direct Acetalisation

792.8 g of C₃₆ dimer alcohol (marketed by the company COGNIS under the designation SPEZIOL® C_{36/2}) are introduced into a two-litre glass reactor, equipped with an efficient mechanical stirring, a system of heating by double envelope, a condenser and a temperature probe.

The dimer alcohol is heated to 90° C. and 112.0 g of xylose are dispersed in the reaction medium and then homogenised at 90°/95° C. for 15 minutes.

1.90 g of 98% sulphuric acid and 1.31 g of 50% hypophosphorous acid are added and the reaction mixture is maintained at 95° C. for 4 hours under partial vacuum, with nitrogen bubbling.

After cooling to 80° C., the mixture is neutralised so as to attain a pH value of a 5% solution of the reaction medium of around 7.1 by adding a solution of sodium borohydride in sodium hydroxide solution.

The resulting product is in the form of a limpid and viscous liquid and has a free alcohol content of 50% by weight.

### EXAMPLE 4

### Demonstration of the Properties of the Compositions in Accordance with the Present Invention as Emulsifying Agents for the Preparation of Vaporisable Oil-In-Water Liquid Emulsions

Emulsions were made by using the products of Examples 1 and 2 as emulsifiers.

Under the experimental conditions used (up to 50% by weight of fatty phases and 10% by weight of emulsifier), all the products of the Examples led to the obtaining of oil-in-water emulsions (determination by the drop method).

These emulsions were all vaporisable, the viscosities being less than 3,000 cps and generally between 100 and 1,500 cps.

### EXAMPLES 5 to 9

### Examples of Vaporisable Oil-In-Water Emulsions Incorporating the Compositions Based on Alkylpolyglycosides and Dimerdiol According to the Invention

### EXAMPLE 5

VAPORISABLE SOLAR EMULSION

| | | | |
|---|---|---|---|
| A) | Composition of Example 1 | | 2% |
| | Octylmethoxycinnamate | | 6% |
| | C₈-C₁₀ triglyceride | | 4% |
| B) | Water | qs | 100% |
| | disodic EDTA | | 0.2% |
| | Xanthan gum | | 0.1% |
| | Perfume | | 0.1% |

The EDTA and the xanthan gum are dispersed in water. The composition of Example 1 is then added and then the various oils. Emulsification is carried out with a turbine rotor stator, the whole at ambient temperature.

Characteristics

Vaporisable lotion: 500 cps

Stability: greater than 1 month at ambient temperature.

### EXAMPLE 6

ALCOHOL-FREE PERFUME SPRAY

| | | | |
|---|---|---|---|
| A) | Composition of Example 2 | | 3% |
| | Perfume concentrate | | 2% |
| | Polyisobutene | | 5% |
| B) | Water | qs | 100% |
| | Micropearl ® M211 | | 2% |
| | SIMULGEL ® EG | | 0.2% |

The constituents of the fatty phase A are pre-mixed. The Simulgel® is added. The Micropearl® is then added, then the water.

Emulsification is carried out with a turbine rotor stator. The whole at ambient temperature.

Characteristics

Vaporisable lotion: 300 cps

Stability: greater than 1 month at ambient temperature.

### EXAMPLE 7

FURNITURE POLISH SPRAY

| | | | |
|---|---|---|---|
| A) | Composition of Example 2 | | 3.5% |
| | linseed oil | | 0.5% |
| | Dimethicone | | 3.5% |
| B) | Water | qs | 100% |

The constituents of the fatty phase A are mixed and then the water is added. Emulsification is carried out with a turbine rotor stator. The whole at ambient temperature.

Characteristics

Vaporisable lotion: 50 cps

Stability: greater than 1 month at ambient temperature.

### EXAMPLE 8

FLUID EMULSION FOR IMPREGNATION OF BABY WIPES

| | | | |
|---|---|---|---|
| A) | Composition of Example 2 | | 2.5% |
| | Liquid paraffin | | 5% |
| B) | Water | qs | 100 |
| | Sorbitol 70% | | 5% |
| C) | Perfume | | 0.10% |
| | Preservatives | | qs |

The sorbitol and the water are mixed, the composition of Example 2, is added, then the liquid paraffin, the perfume and the preservatives.

Emulsification is carried out with a turbine rotor stator at ambient temperature.

Characteristics

Vaporisable lotion: 150 cps

Stability: greater than 1 month at ambient temperature.

### EXAMPLE 9

FLUID EMULSION FOR PULVERISATION OF HANDKERCHIEFS
(MANUFACTURE OF MOISTURISING HANDKERCHIEFS).

| | | | |
|---|---|---|---|
| A) | Composition of Example 2 | | 1% |
| | Calendula oil | | 2% |
| B) | Water | qs | 100% |
| | Propylene glycol | | 5% |

The propylene glycol and the water are mixed, the composition of Example 2 is added, then the oil, emulsification is carried out with a turbine rotor stator, the whole at ambient temperature.

Characteristics

Vaporisable emulsion: 200 cps

Stability: greater than 1 month at ambient temperature.

What is claimed is:

1. Compositions based on alkylpolyglycosides and dimerdiol, which comprise:
5 to 95 parts by weight of a mixture of alkylpolyglycosides which is constituted of the products of a reaction of a saccharide and a dimerdiol having 36 carbon atoms;
95 to 5 parts by weight of a dimerdiol having 36 carbon atoms.

2. Compositions according to claim 1, which comprise:
5 to 60 parts by weight of the above-mentioned mixture of alkylpolyglycosides; and
95 to 40 parts by weight of dimerdiol having 36 carbon atoms.

3. Compositions according to claim 1, wherein the mixture of alkylpolyglycosides mentioned above is constituted of a mixture of any proportion of hydroxyalkylpolyglycosides and of polyglycosylalkylpoly-glycosides which can be represented, respectively, by the following formulae (I) and (II):
HO—R—O(G)ₙ (I)
(G)ₘ—OR—O—(G)ₚ (II)
in which:
G represents a saccharide residue;
R represents a disubstituted group derived from the dimer alcohol originating from the hydrogenation from the dimer acid;
n, m and p represent the average degree of polymerisation of each saccharide residue.

4. Compositions according to claim 2, wherein the mixture of alkylpolyglycosides mentioned above is constituted of a mixture of any proportion of hydroxyalkylpolyglycosides and of polyglycosylalkylpoly-glycosides which can be represented, respectively, by the following formulae (I) and (II):
HO—R—O(G)ₙ (I)
(G)ₘ—OR—O—(G)ₚ (II)
in which:
G represents a saccharide residue;
R represents a disubstituted group derived from the dimer alcohol originating from the hydrogenation from the dimer acid;
n, m and p represent the average degree of polymerisation of each saccharide residue.

5. Compositions according to claim 3, wherein in the formulae (I) and (II) mentioned above:
G represents a glycose residue selected from the group consisting of glucose, dextrose, fructose, galactose, mannose, ribose and xylose; and
n, m and p represent a number between 1.05 and 2.5.

6. Compositions according to claim 3, wherein in the formulae (I) and (II) mentioned above:
G represents a glycose residue selected from the group consisting of glucose, dextrose, fructose, galactose, mannose, ribose and xylose; and
n, m and p represent a number between 1.1 and 2.

7. Compositions according to claim 4, wherein in the formulae (I) and (II) mentioned above:
G represents a glycose residue selected from the group consisting of glucose, dextrose, fructose, galactose, mannose, ribose and xylose; and
n, m and p represent a number between 1.05 and 2.5.

8. Compositions according to claim 4, wherein in the formulae (I) and (II) mentioned above:
G represents a glycose residue selected from the group consisting of glucose, dextrose, fructose, galactose, mannose, ribose and xylose; and
n, m and p represent a number between 1.1 and 2.

9. Vaporisable emulsions of the oil-in-water type comprising an aqueous phase, an oily phase and an emulsifier, wherein said emulsifier is constituted by a composition based on alkylpolyglycosides and dimerdiol as defined according to claim 1.

10. Vaporisable emulsions of the oil-in-water type comprising an aqueous phase, an oily phase and an emulsifier, wherein said emulsifier is constituted by a composition based on alkylpolyglycosides and dimerdiol as defined according to claim 2.

11. Vaporisable emulsions of the oil-in-water type comprising an aqueous phase, an oily phase and an emulsifier, wherein said emulsifier is constituted by a composition based on alkylpolyglycosides and dimerdiol as defined according to claim 3.

12. Vaporisable emulsions of the oil-in-water type comprising an aqueous phase, an oily phase and an emulsifier, wherein said emulsifier is constituted by a composition based on alkylpolyglycosides and dimerdiol as defined according to claim 5.

13. Emulsions according to claim 9, which they comprise:
from 1 to 10% by weight of said composition based on alkylpolyglycosides and dimerdiol;
from 0 to 50% by weight of oil; and
an aqueous phase.

14. Emulsions according to claim 9, which they comprise:
from 1 to 10% by weight of said composition based on alkylpolyglycosides and dimerdiol;
from 2 to 30% by weight of oil; and
an aqueous phase.

15. Emulsions according to claim 10, which they comprise:
from 1 to 10% by weight of said composition based on alkylpolyglycosides and dimerdiol;
from 0 to 50% by weight of oil; and
an aqueous phase.

16. Emulsions according to claim 11, which they comprise:
from 1 to 10% by weight of said composition based on alkylpolyglycosides and dimerdiol;
from 0 to 50% by weight of oil; and
an aqueous phase.

17. Emulsions according to claim 12, which they comprise:
from 1 to 10% by weight of said composition based on alkylpolyglycosides and dimerdiol;
from 0 to 50% by weight of oil; and
an aqueous phase.

18. Emulsions according to claim 10, which they comprise:
from 1 to 10% by weight of said composition based on alkylpolyglycosides and dimerdiol;
from 2 to 30% by weight of oil; and
an aqueous phase.

19. Emulsions according to claim 11, which they comprise:
from 1 to 10% by weight of said composition based on alkylpolyglycosides and dimerdiol;
from 2 to 30% by weight of oil; and
an aqueous phase.

20. Emulsions according to claim 12, which they comprise:
from 1 to 10% by weight of said composition based on alkylpolyglycosides and dimerdiol;
from 2 to 30% by weight of oil; and
an aqueous phase.
